(19) United States
(12) Patent Application Publication
(10) Pub. No.: US 2018/0237459 A1
(43) Pub. Date: Aug. 23, 2018

(54) METHOD FOR COUPLING AN AROMATIC OR VINYLIC COMPOUND TO A BORON-CONTAINING COMPOUND

(71) Applicant: Dow Global Technologies LLC, Midland, MI (US)

(72) Inventors: Patrick S. Hanley, Midland, MI (US); Matthias S. Ober, Midland, MI (US); William J. Kruper, Sanford, MI (US)

(21) Appl. No.: 15/753,248

(22) PCT Filed: Aug. 12, 2016
(86) PCT No.: PCT/US2016/046800

§ 371 Date: Feb. 16, 2018

Related U.S. Application Data
(60) Provisional application No. 62/206,342, filed on Aug. 18, 2015.

(51) Int. Cl. C07F 5/02; B01J 23/755

(52) U.S. Cl. CPC C07F 5/025; C07C 303/08; B01J 23/755

(57) ABSTRACT

In one aspect, there is provided a method of coupling an aromatic or vinylic compound having a fluorosulfonate substituent to a boron-containing compound. In another aspect, there is provided a method of coupling an aromatic or vinylic compound having a hydroxyl substituent to a boron-containing compound in a one-pot reaction.

### BACKGROUND

Miyaura borylation is a valuable synthetic method for coupling an aromatic or a vinylic compound (compounds having an sp² carbon) to a boron-containing compound, thereby forming a new carbon-boron bond between the aromatic or vinylic compound and the boron-containing compound. In one common Miyaura borylation the aromatic or vinylic compound is substituted by a halide. In some instances, the aromatic or vinylic compound used in the Miyaura borylation is prepared from an aromatic or vinylic compound having a hydroxyl substituent.

It is known that triflates, having the formula F₃CSO₃—, may be used in the place of the halides in Miyaura borylations, however the expense of triflic anhydride (CF₃SO₂)₂O has limited the use of triflates in Miyaura borylations to the production of fine chemicals. Further, the atom economy of triflic anhydride is low since half of the molecule is expended as monomeric triflate anion (CF₃SO₃⁻) following functionalization of a phenolic precursor. In some instances Miyaura borylation reactions involving triflates exhibit sensitivity to water under basic conditions.

It is also known that aryl methanesulfonates are suitable for borylation reactions. One drawback of using aryl methanesulfonates is that these reactions require expensive palladium catalysts. Another drawback of using aryl methanesulfonates is low atom economy.

When performing a Miyaura borylation using either a triflate or methanesulfonate, it is common to perform the reaction in two steps, a first step comprising replacing the hydroxyl group on the aromatic or vinylic compound with the triflate or the methanesulfonate, and a second step comprising coupling the aromatic or vinylic compound with the boron-containing compound. A separation step is generally required between the first and second steps.

It would be desirable to have a replacement for triflates and methanesulfonates for the Miyaura borylation.

### STATEMENT OF INVENTION

In one aspect, there is provided a method of coupling an aromatic or vinylic compound having a fluorosulfonate substituent to a boron-containing compound.

In one aspect, there is provided a method of coupling an aromatic or vinylic compound having a hydroxyl substituent to a boron-containing compound in a one-pot reaction.

### DETAILED DESCRIPTION

Unless otherwise indicated, numeric ranges, for instance “from 2 to 10,” are inclusive of the numbers defining the range (e.g., 2 and 10).

Unless otherwise indicated, ratios, percentages, parts, and the like are by weight.

As used herein, unless otherwise indicated, the phrase “molecular weight” refers to the number average molecular weight as measured in conventional manner.

“Alkyl,” as used in this specification, whether alone or as part of another group (e.g., in dialkylamino), encompasses straight and branched chain aliphatic groups having the indicated number of carbon atoms. If no number is indicated (e.g., aryl-alkyl-), then 1-12 alkyl carbons are contemplated. Preferred alkyl groups include, without limitation, methyl, ethyl, propyl, isopropyl, butyl, isobutyl, sec-butyl, tert-butyl, pentyl, hexyl and tert-octyl.

The term “heteroalkyl” refers to an alkyl group as defined above with one or more heteroatoms (nitrogen, oxygen, sulfur, phosphorus) replacing one or more carbon atoms within the radical, for example, an ether or a thioether.

An “aryl” group refers to any functional group or substituent derived from an aromatic ring. In one instance, aryl refers to an aromatic moiety comprising one or more aromatic rings. In one instance, the aryl group is a C₆-C₁₈ aryl group. In one instance, the aryl group is a C₆-C₁₀ aryl group. In one instance, the aryl group is a C₁₀-C₁₈ aryl group. Aryl groups contain 4n+2 pi electrons, where n is an integer. The aryl ring may be fused or otherwise attached to one or more heteroaryl rings, aromatic or non-aromatic hydrocarbon rings or heterocycloalkyl rings. Preferred aryls include, without limitation, phenyl, naphthyl, anthracenyl, and fluorenyl. Unless otherwise indicated, the aryl group is optionally substituted with 1 or more substituents that are compatible with the syntheses described herein. Such substituents include, but are not limited to, sulfonate groups, boron-containing groups, alkyl groups, nitro groups, halogens, cyano groups, carboxylic acids, esters, amides, C₂-C₈ alkene, and other aromatic groups. Other substituents are known in the art. Unless otherwise indicated, the foregoing substituent groups are not themselves further substituted.

“Heteroaryl” refers to any functional group or substituent derived from an aromatic ring and containing at least one heteroatom selected from nitrogen, oxygen, and sulfur. Preferably, the heteroaryl group is a five or six-membered ring. The heteroaryl ring may be fused or otherwise attached to one or more heteroaryl rings, aromatic or non-aromatic hydrocarbon rings or heterocycloalkyl rings. Examples of heteroaryl groups include, without limitation, pyridine, pyrimidine, pyridazine, pyrrole, triazine, imidazole, triazole, furan, thiophene, oxazole, thiazole. The heteroaryl group may be optionally substituted with one or more substituents that are compatible with the syntheses described herein. Such substituents include, but are not limited to, fluorosulfonate groups, boron-containing groups, C₁-C₈ alkyl groups, nitro groups, halogens, cyano groups, carboxylic acids, esters, amides, C₂-C₈ alkene and other aromatic groups. Other substituents are known in the art. Unless otherwise indicated, the foregoing substituent groups are not themselves further substituted.

“Aromatic compound” refers to a ring system having 4n+2 pi electrons where n is an integer. “Vinylic compound” refers to an alkene.

As noted above, the present disclosure describes a process for coupling an aromatic or vinylic compound to a boron-containing compound. This process is shown generally in Equation 1, whereby an aromatic or vinylic compound having a hydroxyl group is first reacted with SO₂F₂ and a base and is second reacted with a boron-containing compound in the presence of a catalyst. It is understood that where a hydroxyl group is indicated, the hydroxyl group could be deprotonated to form a phenolate (e.g. the deprotonation step could be performed prior to introduction of A to the reaction mixture or after the introduction to the reaction mixture).

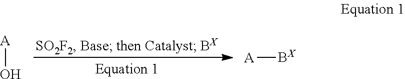

Unexpectedly, it has been found that the reaction of Equation 1 may be performed as a one-pot reaction, as compared to performing the reaction indiscrete steps. Without being limited by theory, it is anticipated that the reaction shown in Equation 1 proceeds along the same reaction path whether performed as a one-pot reaction or as discrete steps. When performed in discrete steps, the first step comprises reacting an aromatic or vinylic compound having a hydroxyl substituent with SO₂F₂ to yield the product shown in Equation 2, and the second step comprises reacting the product of Equation 2 with a boron-containing compound to yield the product shown in Equation 3.

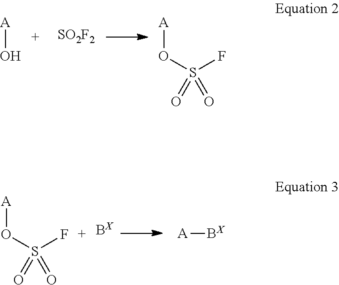

In one instance, the process involves a one-pot reaction where an aromatic or vinylic compound (it is understood that the vinylic compound may be formed from another compound, such as an aldehyde, ester or ketone) having a hydroxyl group is first reacted with SO₂F₂ and a base and is second reacted with a boron-containing compound in the presence of a catalyst, as shown generally in Equation 1. Without being limited by theory, it is expected that Equation 3 is the same general reaction as depicted by step 2) of the reaction shown in Equation 1.

As used in Equation 1, Equation 2 and Equation 3, the aromatic or vinylic compound is identified as A and the boron-containing compound is identified as B^{X}. The aromatic or vinylic compound is either an aryl group or a heteroaryl group. The boron-containing compound is a boron-containing substituent known to be suitable for use in Miyaura borylation, for example, bis(pinacolato)diboron (B₂pin₂), B₂OH₄, as identified in Equation 1 and Equation 3 as B^{x}. The result of the reactions shown in Equation 1 and Equation 3 is the formation of a new carbon-boron bond between the aromatic or vinylic compound and the boron-containing compound.

As noted above in the first step of Equation 1 and in Equation 2, the aromatic or vinylic compound is bonded to a fluorosulfonate group. A fluorosulfonate group refers to O-fluorosulfonate of the formula —OSO₂F. O-fluorosulfonate may be synthesized from sulfuryl fluoride. The fluorosulfonate group serves as a leaving group from the aromatic or vinylic compound. Without being limited by theory, the sulfuryl atom of the fluorosulfonate group is bonded to the oxygen of the hydroxyl group of the aromatic or vinylic compound.

As noted above, the boron-containing compound includes a boron-containing substituent known to be suitable for use in Miyaura borylation, for example, bis(pinacolato)diboron (B₂pin₂), B₂OH₄, as identified in Equation 1 and Equation 3 as B^{x}. Preferably, the boron-containing compound is a compound having a boron-boron bond, and each boron is bonded to two oxygens. The oxygens may be alcohols or may include other substituents.

As noted above in Equation 1 and Equation 3, the aromatic or vinylic compound is reacted with the boron-containing compound in a reaction mixture. The reaction mixture includes a catalyst having at least one group 10 atom. In some instances, the reaction mixture also includes a ligand, and a base. The group 10 atoms include nickel, palladium and platinum.

The catalyst is provided in a form suitable to the reaction conditions. In one instance, the catalyst is provided on a substrate. In one instance, the catalyst having at least one group 10 atom is generated in situ from one or more precatalysts and one or more ligands. Examples of palladium precatalysts include, but are not limited to, Palladium(II) acetate, Palladium(II) chloride, Dichlorobis(acetonitrile)palladium(II), Dichlorobis(benzonitrile)palladium(II), Allylpalladium chloride dimer, Palladium(II) acetylacetonate, Palladium(II) bromideBis(dibenzylideneacetone)palladium(0), Bis(2-methylallyl)palladium chloride dimer, Crotylpalladium chloride dimer, Dichloro(1,5-cyclooctadiene)palladium(II), Dichloro(norbornadiene)palladium(II), Palladium(II) trifluoroacetate, Palladium(II) benzoate, Palladium(II) trimethylacetate, Palladium(II) oxide, Palladium(II) cyanide, Tris(dibenzylideneacetone)dipalladium(0), Palladium(II) hexafluoroacetylacetonate, cis-Dichloro(N,N,N′,N′-tetramethylethylenediamine) palladium(II), and Cyclopentadienyl[(1,2,3-n)-1-phenyl-2-propenyl]palladium(II).

In one instance, nickel-based catalysts are used. In another instance, platinum-based catalysts are used. In yet another instance, a catalyst including one or more of nickel, platinum and palladium-based catalysts are used.

In one instance, pyridine-enhanced precatalyst preparation stabilization and initiation (PEPPSI) type catalysts are used, for example, [1,3-Bis(2,6-Diisopropylphenyl)imidazol-2-ylidene](3-chloropyridyl)palladium(II) dichloride, and (1,3-Bis(2,6-diisopropylphenyl)imidazolidene) (3-chloropyridyl) palladium(II) dichloride.

Examples of nickel precatalysts include, but are not limited to, nickel(II) acetate, nickel(II) chloride, Bis(triphenylphosphine)nickel(II) dichloride, Bis(tricyclohexylphosphine)nickel(II) dichloride, [1,1′-Bis(diphenylphosphino)ferrocene]dichloronickel(II), Dichloro[1,2-bis(diethylphosphino)ethane]nickel(II), Chloro(1-naphthyl)bis(triphenylphosphine)nickel(II), 1,3-Bis(2,6-diisopropylphenyl)imidazolium chloride, Bis(1,5-cyclooctadiene)nickel(0), Nickel(II) chloride ethylene glycol dimethyl ether complex, [1,3-Bis(diphenylphosphino)propane]dichloronickel(II), [1,2-Bis(diphenylphosphino)ethane]dichloronickel(II), and Bis(tricyclohexylphosphine)nickel(0).

The ligand used in the reaction mixture is preferably selected to generate the selected catalyst from a pre-catalyst. For example, the ligand may be a phosphine ligand, a carbene ligand, an amine-based ligand, a carboxylate based ligand, an aminodextran, an aminophosphine-based ligands or an N-heterocyclic carbene-based ligand. In one instance, the ligand is monodentate. In one instance, the ligand is bidentate. In one instance, the ligand is polydentate.

Suitable phosphine ligands may include, but are not limited to, mono- and bi-dentate phosphines containing functionalized aryl or alkyl substituents or their salts. For example, suitable phosphine ligands include, but are not limited to, triphenylphosphine; Tri(o-tolyl)phosphine; Tris(4-methoxyphenyl)phosphine; Tris(pentafluorophenyl)phosphine; Tri(p-tolyl)phosphine; Tri(2-furyl)phosphine; Tris(4-chlorophenyl)phosphine; Di(1-adamantyl)(1-naphthoyl)phosphine; Benzyldiphenylphosphine; 1,1′-Bis(di-t-butylphosphino)ferrocene; (−)-1,2-Bis((2R,5R)-2,5-dimethylphospholano)benzene; (−)-2,3-Bis[(2R,5R)-2,5-dimethylphospholanyl]-1-[3,5-bis(trifluoromethyl)phenyl]-1H-pyrrole-2,5-dione; 1,2-Bis(diphenylphosphino)benzene; 2,2′-Bis(diphenylphosphino)-1,1′-binaphthyl; 2,2′-Bis(diphenylphosphino)-1,1′-biphenyl, 1,4-Bis(diphenylphosphino)butane; 1,2-Bis(diphenylphosphino)ethane; 2-[Bis(diphenylphosphino)methyl]pyridine; 1,5-Bis(diphenylphosphino)pentane; 1,3-Bis(diphenylphosphino)propane; 1,1′-Bis(di-i-propylphosphino)ferrocene; (S)-(−)-5,5′-Bis[di(3,5-xylyl)phosphino]-4,4′-bi-1,3-benzodioxole; tricyclohexylphosphine (referred to herein as PCy3); Tricyclohexylphosphine tetrafluoroborate (referred to herein as PCy3.HBF₄); N-[2-(di-1-adamantylphosphino) phenyl]morpholine; 2-(Di-t-butylphosphino)biphenyl; 2-(Di-t-butylphosphino)-3,6-dimethoxy-2′,4′,6′-tri-i-propyl-1,1′-biphenyl; 2-Di-t-butylphosphino-2′-(N,N-dimethylamino)biphenyl; 2-Di-t-butylphosphino-2′-methylbiphenyl; Dicyclohexylphenylphosphine; 2-(Dicyclohexylphosphino)-3,6-dimethoxy-2′,4′,6′-tri-i-propyl-1; 2-(Dicyclohexylphosphino)-2′-(N,N-dimethylamino)biphenyl; 2-Dicyclohexylphosphino-2′,6′-dimethylamino-1,1′-biphenyl; 2-Dicyclohexylphosphino-2′,6′-di-i-propoxy-1,1′-biphenyl; 2-Dicyclohexylphosphino-2′-methylbiphenyl; 2-Dicyclohexylphosphino-2′,4′,6′-triisopropylbiphenyl; 2-[2-(Dicyclohexylphosphino)phenyl]-1-methyl-1H-indole; 2-(Dicyclohexylphosphino)-2′,4′,6′-tri-i-propyl-1,1′-biphenyl; [4-(N,N-Dimethylamino)phenyl]di-t-butylphosphine; 9,9-Dimethyl-4,5-bis(diphenylphosphino)xanthene; (R)-(−)-1-[(S)-2-(Diphenylphosphino)ferrocenyl]ethyldicyclohexylphosphine; Tribenzylphosphine; Tri-t-butylphosphine; Tri-n-butylphosphine; and 1,1′-Bis(diphenylphosphino)ferrocene (referred to herein as “DPPF”).

Suitable amine and aminophosphine-based ligands include any combination of monodentate or bidentate alkyl and aromatic amines including, but not limited to, pyridine, 2,2′-Bipyridyl, 4,4′-Dimethyl-2,2′-dipyridyl, 1,10-Phenanthroline, 3,4,7,8-Tetramethyl-1,10-phenanthroline, 4,7-Dimethoxy-1,10-phenanthroline, N,N,N′,N′-Tetramethylethylenediamine, 1,3-Diaminopropane, ammonia, 4-(Aminomethyl)pyridine, (1R,2S,9S)-(+)-11-Methyl-7,11-diazatricyclo[7.3.1.0²′7]tridecane, 2,6-Di-tert-butylpyridine, 2,2′-Bis[(4S)-4-benzyl-2-oxazoline], 2,2-Bis((4S)-(−)-4-isopropyloxazoline)propane, 2,2′-Methylenebis[(4S)-4-phenyl-2-oxazoline], and 4,4′-di-tert-butyl-2,2′-bipyridyl. In addition, aminophosphine ligands such as 2-(Diphenylphosphino)ethylamine, 2-(2-(Diphenylphosphino)ethyl)pyridine, (1R,2R)-2-(diphenylphosphino)cyclohexanamine, an aminodextran and 2-(Di-tert-butylphosphino)ethylamine.

Suitable carbene ligands include N-heterocyclic carbene (NHC) based ligands, including, but not limited to, 1,3-Bis(2,4,6-trimethylphenyl)imidazolinium chloride, 1,3-Bis(2,6-diisopropylphenyl)imidazolium chloride, 1,3-Bis-(2,6-diisopropylphenyl) imidazolinium chloride, 1,3-Diisopropylimidazolium chloride, and 1,3-Dicyclohexylbenzimidazolium chloride.

The base used in the reaction mixture is selected to be compatible with the catalyst, the boron-containing compound and the fluorosulfonate. Suitable bases include, but are not limited to, carbonate salts, phosphate salts, acetate salts and carboxylic acid salts. Unexpectedly, it has been found that inorganic bases are suitable in the reaction mixture.

Examples of carbonate salts include, but are not limited to, lithium carbonate, sodium carbonate, potassium carbonate, rubidium carbonate, cesium carbonate, ammonium carbonate, substituted ammonium carbonates, and the corresponding hydrogen carbonate salts. Examples of phosphate salts include, but are not limited to, lithium phosphate, sodium phosphate, potassium phosphate, rubidium phosphate, cesium phosphate, ammonium phosphate, substituted ammonium phosphates, and the corresponding hydrogen phosphate salts. Examples of acetate salts include, but are not limited to, lithium acetate, sodium acetate, potassium acetate, rubidium acetate, cesium acetate, ammonium acetate, and substituted ammonium acetates.

Other bases include, but are not limited to, salts of formate, fluoroacetate, and propionate anions with lithium, sodium, potassium, rubidium, cesium, ammonium, and substituted ammonium cations; metal hydroxides, such as lithium hydroxide, sodium hydroxide, potassium hydroxide, metal dihydroxides such as magnesium dihydroxide, calcium dihydroxide, strontium dihydroxide, and barium dihydroxide; metal trihydroxides such as aluminum trihydroxide, gallium trihydroxide, indium trihydroxide, thallium trihydroxide; non nucleophilic organic amines such as triethylamine, N,N-diisopropylethylamine, 1,4-diazabicyclo[2.2.2]octane (DABCO), 1,5-Diazabicyclo[4.3.0]non-5-ene (DBN), 1,8-Diazabicyclo[5.4.0]undec-7-ene (DBU); bis(silyl)amide salts such as the lithium, sodium, and potassium salts of bis(trimethylsilyl)amide; alkoxide salts such as the lithium, sodium, and potassium salts of t butoxide; and 1,8-bis(dimethylamino) naphthalene; metal fluorides, such as sodium fluoride, potassium fluoride, cesium fluoride, silver fluoride, tetra butyl ammonium fluoride, ammonium fluoride, triethyl ammonium fluoride.

Examples of amine bases, such as alkylamines and heteroarenes include, but are not limited to, triethylamine, pyridine, morpholine, 2,6-lutidine, triethylamine, N,N-Dicyclohexylmethylamine, and diisopropylamine.

In one instance, the base is used in the presence of a phase-transfer catalyst. In another instance, the base is used in the presence of water. In yet another instance, the base is used in the presence of an organic solvent. In still another instance, the base is used in the presence of one or more of a phase-transfer catalyst, water or an organic solvent.

Preferably, at least one equivalent of base is present for each equivalent of fluorosulfonate. In some embodiments, no more than 10 equivalents of base are present for each equivalent of fluorosulfonate. In some embodiments, at least 2 equivalents of base are present for each equivalent of fluorosulfonate. In some embodiments, no more than 6 equivalents of base are present for each equivalent of fluorosulfonate.

The solvent in the reaction mixture is selected such that it is suitable for use with the reactants, the catalyst, the ligand and the base. For example, suitable solvents include toluene, xylenes (ortho-xylene, meta-xylene, para-xylene or mixtures thereof), benzene, water, methanol, ethanol, 1-propanol, 2-propanol, n-butanol, 2-butanol, pentanol, hexanol, tert-butyl alcohol, tert-amyl alcohol, ethylene glycol, 1,2-propanedioal, 1,3-propanediol, glycerol, N-methyl-2-pyrrolidone, acetonitrile, N,N-dimethylformamide, methyl acetate, ethyl acetate, propyl acetate, isopropyl acetate, triacetin, acetone, methyl ethyl ketone, and ethereal solvents, such as 1,4-dioxane, tetrahydrofuran, 2-methyltetrahydrofuran, diethylether, cyclopenyl methyl ether, 2-butyl ethyl ether, dimethoxyethane, polyethyleneglycol. In one instance, the solvent includes any combination of the solvents described herein, in, or in the absence of, a surfactant. In one instance, the sulfuryl fluoride is used neat at a sufficiently low temperature that the sulfuryl fluoride is in a liquid.

In one instance, water is included in the reaction mixture. One benefit of using fluorosulfonates as compared to triflates, is that the reaction can be carried out without a subsequent separation step, or with a simple separation step. In couplings involving triflates, a dedicated purification step is required to remove byproducts since the products and the byproducts typically occupy the same phase. In the reaction schemes described herein, the byproducts are either in the gas phase, and will bubble out spontaneously or with a simple degassing step, or will partition into the aqueous phase, which is easily separable. As such, the reaction scheme described herein provides additional benefits as compared to couplings involving triflates.

In one instance, the reaction described herein is completed as a one-pot reaction as shown in Equation 1. In a first step, an aromatic or vinylic compound having an alcohol substituent is added to a reaction mixture in the presence of sulfuryl fluoride and a base (it is understood that the vinylic compound may be in equilibrium with other forms of the compound, for example, ketones, esters, and aldehydes). The base may be any of the bases described herein, including, without limitation, amine bases and inorganic bases. This first step couples the fluorosulfonate substituent to the oxygen of the hydroxyl group. To the reaction mixture formed during this first step is added a boron-containing compound and a catalyst. The catalyst may be a suitable group 10 catalyst, including, without limitation, platinum, palladium and nickel catalysts. The product of this second step is a compound formed by coupling the aromatic or vinylic compound and the boron-containing compound.

Some embodiments of the invention will now be described in detail in the following Examples. Unless stated otherwise, reported yields are ±5%.

### Example 1. Preparation of 4,4,5,5-tetramethyl-2-(p-tolyl)-1,3,2-dioxaborolane

This Example describes the preparation of 4,4,5,5-tetramethyl-2-(p-tolyl)-1,3,2-dioxaborolane as illustrated in Equation 4.

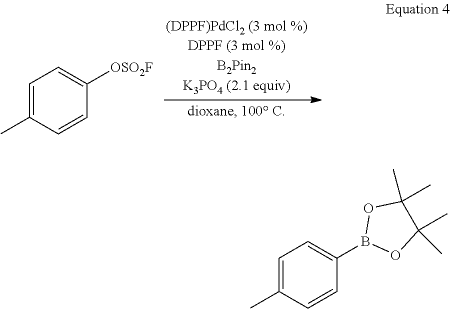

To a 30 mL vial is added p-tolylfluorosulfonate (0.295 g), (DPPF) PdCl₂ (0.032 g) DPPF (0.024 g) potassium phosphate (0.669 g), and b₂pin₂ (0.390 g). To the mixture is added dioxane (3 mL) and the mixture is heated to 100° C. for 19 hours. Gas chromatography-mass spectrometry analysis indicates the formation of the desired product. The reaction mixture is washed with water (5 mL) and extracted with ethyl acetate (2×15 mL). The combined organic layers are dried over MgSO₄, and volatile removed by vacuum. The product is purified by flash chromatography (hexane:ethyl acetate 0-40% gradient). The product, 4,4,5,5-tetramethyl-2-(p-tolyl)-1,3,2-dioxaborolane, is collected as a colorless oil (0.226 g, 69% yield). The identity of the product is confirmed by ¹H NMR (400 MHz, Chloroform-d): δ 7.71 (d, J=7.8 Hz, 2H), 7.19 (dd, J=7.5, 0.7 Hz, 2H), 2.37 (s, 3H), 1.34 (s, 12H). ¹³C NMR (101 MHz, cdcl₃) δ 141.4, 134.8, 128.5, 83.9, 24.8, 21.7, which matches reported NMR spectra for this compound.

### Example 2. One-Pot Preparation of Ethyl 4-(4,4,5,5-tetramethyl-1,3,2-dioxaborolan-2-yl)benzoate

This Example describes the one-pot preparation of Ethyl 4-(4,4,5,5-tetramethyl-1,3,2-dioxaborolan-2-yl)benzoate as illustrated in Equation 5.

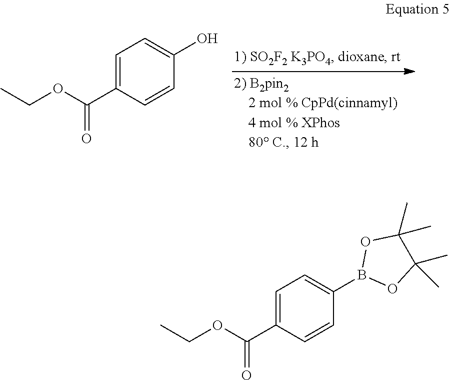 [SMILES: CCOC(=O)c1ccc(O)cc1 ; CCOC(=O)c1ccc(B2OC(C)(C)C(C)(C)O2)cc1]

To a 30 mL scintillation vial is added ethyl-4-hydroxybenzoate (0.415 g; 2.50 mmol) and potassium phosphate (1.65 g; 7.75 mmol). To the mixture is added 6 mL of dioxane and the mixture is stirred vigorously with a magnetic stirbar. Sulfuryl fluoride was slowly bubbled through the reaction mixture for 36 hours. The mixture is degassed by bubbling N₂ through the mixture for 15 mins. In an N₂ filled glovebox, bis(pinacolato)diboron (0.635, 2.5 mmol), 2-Dicyclohexylphosphino-2′,4′,6′-triisopropylbiphenyl (XPhos) (0.048 g; 0.10 mmol), CpPd(cinnamyl) (0.014 g; 0.05 mmol), and an additional 1 mL of dioxane are added to the mixture. The reaction is heated to 80° C. and stirred for 12 hours. The desired product is purified by flash chromatography (hexane/ethyl acetate). The product, Ethyl 4-(4,4,5,5-tetramethyl-1,3,2-dioxaborolan-2-yl)benzoate, is collected as a light brown oil (0.385 g, 56% yield). The identity of the product is confirmed by ¹H NMR (400 MHz, Chloroform-d) δ 8.02 (d, J=8.4 Hz, 2H), 7.86 (d, J=8.4 Hz, 2H), 4.38 (q, J=7.2 Hz, 2H), 1.40 (t, J=7.2 Hz, 3H), 1.36 (s, 12H). ¹³C NMR (101 MHz, CDCl3) δ 166.7, 134.6, 132.7, 128.5, 84.2, 61.0, 24.9, 14.3, which matches reported NMR spectra for this compound.

What is claimed is:

1. A method of coupling an aromatic or vinylic compound to a boron-containing compound, the method comprising:
providing the aromatic or vinylic compound having a fluorosulfonate substituent;
providing the boron-containing compound; and
reacting the aromatic or vinylic compound and the boron-containing compound in a reaction mixture, the reaction mixture including a catalyst having at least one group 10 atom, the reaction mixture under conditions effective to couple the aromatic or vinylic compound to the boron-containing compound thereby forming a new carbon-boron bond between the aromatic or vinylic compound and the boron-containing compound.

2. The method of claim 1, wherein the reaction mixture further includes a ligand, and a base.

3. The method of claim 1, wherein the aromatic or vinylic compound is heteroaryl.

4. The method of claim 1 wherein the catalyst is a palladium catalyst or a nickel catalyst.

5. The method of claim 4, wherein the catalyst is generated in-situ from a palladium precatalyst, the palladium precatalyst is selected from the group consisting of: Palladium(II) acetate, Palladium(II) chloride, Dichlorobis(acetonitrile)palladium(II), Dichlorobis(benzonitrile)palladium(II), Allylpalladium chloride dimer, Palladium(II) acetylacetonate, Palladium(II) bromide, Bis(dibenzylideneacetone)palladium(0), Bis(2-methylallyl)palladium chloride dimer, Crotylpalladium chloride dimer, Dichloro(1,5-cyclooctadiene)palladium(II), Dichloro(norbornadiene)palladium(II), Palladium(II) trifluoroacetate, Palladium(II) benzoate, Palladium(II) trimethylacetate, Palladium(II) oxide, Palladium(II) cyanide, Tris(dibenzylideneacetone)dipalladium(0), Palladium(II) hexafluoroacetylacetonate, cis-Dichloro(N,N,N′,N′-tetramethylethylenediamine)palladium(II), Cyclopentadienyl[(1,2,3-n)-1-phenyl-2-propenyl]palladium(II), [1,3-Bis(2,6-Diisopropylphenyl)imidazol-2-ylidene](3-chloropyridyl)palladium(II) dichloride, (1,3-Bis(2,6-diisopropylphenyl)imidazolidene) (3-chloropyridyl) palladium(II) dichloride, and a mixture of two or more thereof.

6. The method of claim 4, wherein the catalyst is generated in-situ from a nickel precatalyst, the nickel precatalyst is selected from the group consisting of: nickel(II) acetate, nickel(II) chloride, Bis(triphenylphosphine)nickel(II) dichloride, Bis(tricyclohexylphosphine)nickel(II) dichloride, [1,1′-Bis(diphenylphosphino)ferrocene]dichloronickel(II), Dichloro[1,2-bis(diethylphosphino)ethane]nickel(II), Chloro(1-naphthyl)bis(triphenylphosphine) nickel(II), 1,3-Bis(2,6-diisopropylphenyl)imidazolium chloride, Bis(1,5-cyclooctadiene)nickel(0), Nickel(II) chloride ethylene glycol dimethyl ether complex, [1,3-Bis(diphenylphosphino)propane]dichloronickel(II), [1,2-Bis(diphenylphosphino)ethane]dichloronickel(II), Bis(tricyclohexylphosphine)nickel(0).

7. The method of claim 2 wherein the ligand includes one or more of a phosphine ligand, a carbene ligand, an amine-based ligand, an aminophosphine-based ligand.

8. The method of claim 2, wherein the base is a carbonate salt, a phosphate salt, an acetate salt or a carboxylic acid salt.

9. The method of claim 2, wherein the base is selected from the group consisting of lithium carbonate, sodium carbonate, potassium carbonate, rubidium carbonate, cesium carbonate, ammonium carbonate, substituted ammonium carbonates, hydrogen carbonates, lithium phosphate, sodium phosphate, potassium phosphate, rubidium phosphate, cesium phosphate, ammonium phosphate, substituted ammonium phosphates, hydrogen phosphates, lithium acetate, sodium acetate, potassium acetate, rubidium acetate, cesium acetate, ammonium acetate, substituted ammonium acetates, formate salts, fluoroacetate salts, propionate anions with lithium, sodium, potassium, rubidium, cesium, ammonium, and substituted ammonium cations, lithium hydroxide, sodium hydroxide, potassium hydroxide, magnesium dihydroxide, calcium dihydroxide, strontium dihydroxide, and barium dihydroxide, aluminum trihydroxide, gallium trihydroxide, indium trihydroxide, thallium trihydroxide, triethylamine, N,N-diisopropylethylamine, 1,4-diazabicyclo[2.2.2]octane, 1,5-Diazabicyclo[4.3.0]non-5-ene, 1,8-Diazabicyclo[5.4.0]undec-7-ene, lithium, sodium, and potassium salts of bis(trimethylsilyl)amide, lithium, sodium, and potassium salts of t butoxide, 1,8-bis(dimethylamino)naphthalene, pyridine, morpholine, 2,6-lutidine, triethylamine, N,N-Dicyclohexylmethylamine, diisopropylamine, sodium fluoride, potassium fluoride, cesium fluoride, silver fluoride, tetra butyl ammonium fluoride, ammonium fluoride, triethyl ammonium fluoride and a mixture of two or more thereof.

10. The method of claim 1, wherein the reaction mixture includes a solvent.

11. The method of claim 10, wherein the solvent is selected from the group consisting of toluene, xylenes (ortho-xylene, meta-xylene, para-xylene or mixtures thereof), benzene, water, methanol, ethanol, 1-propanol, 2-propanol, n-butanol, 2-butanol, pentanol, hexanol, tert-butyl alcohol, tert-amyl alcohol, ethylene glycol, 1,2-propanedioal, 1,3-propanediol, glycerol, N-methyl-2-pyrrolidone, acetonitrile, N,N-dimethylformamide, methyl acetate, ethyl acetate, propyl acetate, isopropyl acetate, triacetin, acetone, methyl ethyl ketone, and ethereal solvents, such as 1,4-dioxane, tetrahydrofuran, 2-methyltetrahydrofuran, diethylether, cyclopentyl methyl ether, 2-butyl ethyl ether, dimethoxyethane, and polyethyleneglycol.

12. The method of claim 1, wherein the boron-containing compound is a boron-containing compound is a compound having a boron-boron bond, and each boron is bonded to two oxygens.

13. A method of coupling an aromatic or vinylic compound to a boron-containing compound, the method comprising:
providing the aromatic or vinylic compound having a hydroxyl substituent;
providing sulfuryl fluoride in the presence of a base;
reacting the aromatic or vinylic compound and the sulfuryl fluoride in a reaction mixture, the reaction mixture under conditions effective to couple the sulfur atom of the sulfuryl fluoride to the oxygen of the hydroxyl group;
providing to the reaction mixture the boron-containing compound;
providing to the reaction mixture a catalyst having at least one group 10 atom; and
reacting the aromatic or vinylic compound and the boron-containing compound in the reaction mixture, the reaction mixture under conditions effective to couple the aromatic or vinylic compound to the boron-containing compound.

14. The method of claim 13, the base comprising an inorganic base.

15. The method of claim 14, the base comprising an amine base.

16. The method of claim 13, the catalyst comprising a group 10 catalyst.

17. The method of claim 16, the catalyst comprising a nickel-based catalyst.
